(19) United States
(12) Patent Application Publication
(10) Pub. No.: US 2024/0252503 A1
(43) Pub. Date: Aug. 1, 2024

(54) METHODS OF TREATMENT

(71) Applicant: Arena Pharmaceuticals, Inc., New York, NY (US)

(72) Inventors: Brandon James Walsh, La Jolla, CA (US); John William Adams, La Jolla, CA (US)

(73) Assignee: Arena Pharmaceuticals, Inc., New York, NY (US)

(21) Appl. No.: 18/565,626

(22) PCT Filed: May 27, 2022
(86) PCT No.: PCT/US2022/031452

§ 371 Date: Nov. 30, 2023

Related U.S. Application Data
(60) Provisional application No. 63/195,443, filed on Jun. 1, 2021.

(51) Int. Cl. A61K 31/5377; A61K 9/48; A61P 9/10

(52) U.S. Cl. CPC A61K 31/5377; A61K 9/4825; A61P 9/10

(57) ABSTRACT

Provided are methods of treating or preventing Raynaud's, comprising prescribing and/or administering to an individual in need thereof 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof.

Provided are methods useful in the treatment of Raynaud's.

Raynaud's (also referred to as Raynaud/Raynaud's syndrome, Raynaud/Raynaud's disease, and/or Raynaud/Raynaud's phenomenon) is a condition in which vasospasm of the arteries reduces blood flow. During an “attack,” the vessels narrow and limit blood circulation to affected areas, typically involving the fingers and occasionally the toes, nose, or ears. Raynaud's is triggered by the sympathetic nervous system, cold exposure, and/or emotional stress. The most severe complication is critical digital ischemia that may lead to digital ulceration and occasionally gangrene.

Raynaud's presents in primary and secondary forms. The cause of primary Raynaud's is not known. Vascular abnormality with this form is thought to be purely functional and reversible without progressing to irreversible tissue injury. However, some cases of primary Raynaud's progress to secondary Raynaud's.

Secondary Raynaud's occurs as a result of another condition, such as systemic sclerosis (also referred to as scleroderma or systemic scleroderma), rheumatoid arthritis, lupus, vasculitis, atherosclerosis, cryoglobulinemia, hypothyroidism, injury, and/or drug reaction. For example, over 95% of individuals with systemic sclerosis also present with Raynaud's. The severity of secondary Raynaud's is related to the occurrence of structural and functional vascular abnormalities.

Raynaud's is complex and likely involves an interplay between vascular factors, neural control mechanisms, and intravascular factors. The treatment approach for Raynaud's is consistent in mild cases, but diverges in cases involving moderate to severe digital ulceration or underlying systemic sclerosis. Intravenous prostanoids (prostacyclins and analogues) are used in individuals who develop ischaemic digital complications. Intravenous iloprost has been shown to reduce weekly Raynaud phenomenon attacks (39.1% versus 22.2%) and improve severity score (34.8% versus 19.7%) over 9 weeks. Wigley 1994 Ann Intern Med. 1994 Feb. 1; 120(3):199-206. Intravenous epoprostenol has been associated with reduced frequency and duration of Raynaud phenomenon attacks, but loss in clinical response was observed 8-10 weeks after the last infusion. Belch 1983 Lancet. 1(8320):313e5.

Even if intravenous administration were efficacious, it is inconvenient and not feasible for long-term treatment. Further, oral prostanoids are often poorly tolerated and offer little or no significant benefit. In a recent survey, only 16% of individuals found current medication for Raynaud's to be effective. Hughes et al. Rheum 2015. 54 (8): 1443-1447.

5-HT2A antagonism been evaluated for Reynaud's, but clinical results have been mixed.

There are currently no approved drugs for the treatment of Raynaud's. New compounds for the treatment of Raynaud's are needed.

### SUMMARY

Provided herein is a method of treating or preventing Raynaud's, comprising:
prescribing and/or administering to an individual in need thereof 3-methoxy-N-[3-(2-methylpyrazol-3-yl)-4-(2-morpholin-4-ylethoxy)phenyl]benzamide (Compound 1) or a pharmaceutically acceptable salt, hydrate, or solvate thereof.

Also provided herein is the use of 3-methoxy-N-[3-(2-methylpyrazol-3-yl)-4-(2-morpholin-4-ylethoxy)phenyl]benzamide (Compound 1) or a pharmaceutically acceptable salt, hydrate, or solvate thereof in the manufacture of a medicament for the treatment of Raynaud's in an individual in need thereof.

Also provided herein is 3-methoxy-N-[3-(2-methylpyrazol-3-yl)-4-(2-morpholin-4-ylethoxy)phenyl]benzamide (Compound 1) or a pharmaceutically acceptable salt, hydrate, or solvate thereof for use in a method of treatment of Raynaud's in an individual in need thereof.

Also provided herein is 3-methoxy-N-[3-(2-methylpyrazol-3-yl)-4-(2-morpholin-4-ylethoxy)phenyl]benzamide (Compound 1) or a pharmaceutically acceptable salt, hydrate, or solvate thereof in the treatment of Raynaud's in an individual in need thereof.

In some embodiments, the Compound 1, or a pharmaceutically acceptable salt, hydrate, or solvate thereof, is a pharmaceutically acceptable salt of Compound 1.

In some embodiments, the Compound 1, or a pharmaceutically acceptable salt, hydrate, or solvate thereof, is the HCl salt of Compound 1.

These and other aspects of the invention will be apparent upon reference to the following detailed description. To this end, various references are set forth herein which describe in more detail certain background information, procedures, compounds, and/or compositions, and are each hereby incorporated by reference in their entirety.

### DETAILED DESCRIPTION

As used in the present specification, the following words and phrases are generally intended to have the meanings as set forth below, except to the extent that the context in which they are used indicates otherwise.

COMPOUND 1: As used herein, “Compound 1” refers to 3-methoxy-N-[3-(2-methylpyrazol-3-yl)-4-(2-morpholin-4-ylethoxy)phenyl]benzamide, including crystalline forms thereof. As a non-limiting example, Compound 1 may be present in the crystalline form disclosed in U.S. Pat. No. 8,980,891 (incorporated by reference herein in its entirety), which may be characterized by one or more of the following °2θ values for the peaks in the PXRD spectrum with CuKα radiation: 5.0998, 18.7064, 19.1157, 19.3029, and 23.6567, wherein the reported peaks can vary by about ±0.2 °2θ.

Compound 1, or a pharmaceutically acceptable salt, hydrate, or solvate thereof, and its active metabolites (M1 and M2) have a high affinity for human 5-HT2A with no appreciable affinity for 5-HT_{2B} and 5-HT_{2C} receptors. M1 refers to N-(4-(2-(2-hydroxyethylamino)ethoxy)-3-(1-methyl-1H-pyrazol-5-yl)phenyl)-3-methoxybenzamide. M2 refers to N-(4-(2-aminoethoxy)-3-(1-methyl-1H-pyrazol-5-yl)phenyl)-3-methoxybenzamide.

TREAT, TREATING, OR TREATMENT: As used herein the term “treat,” “treating,” or “treatment” refers to the administration of therapy to an individual (i.e., a human) who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with a particular disorder. Therefore, treating a disorder does not necessarily mean a reduction in severity of all symptoms associated with a disorder and does not necessarily mean a complete reduction in the severity of one or more symptoms associated with a disorder.

PREVENT, PREVENTING, OR PREVENTION: As used herein, the term “prevent,” “preventing” or “prevention” means prevention of the occurrence or onset of one or more symptoms associated with a particular disorder and does not necessarily mean the complete prevention of a disorder. For example, the term “prevent,” “preventing” and “prevention” refers to the administration of therapy on a prophylactic or preventative basis to an individual who may ultimately manifest at least one symptom of a disease or condition but who has not yet done so. Such individuals can be identified since risk factors that are known to correlate with the subsequent occurrence of the disease. Alternatively, prevention therapy can be administered without prior identification of a risk factor, as a prophylactic measure. Delaying the onset of the at least one symptom can also be considered prevention or prophylaxis.

DOSE: As used herein, a “dose” means the measured quantity of an active agent to be taken at one time by an individual. In certain embodiments, wherein the active agent is not the free base of Compound 1, the quantity is the molar equivalent to the corresponding amount of the free base of Compound 1. For example, often a drug is packaged in a pharmaceutically acceptable salt form, for example the HCl salt of Compound 1, and the dosage for strength refers to the mass of the molar equivalent of the corresponding free base of Compound 1.

TOLERATE: As used herein, an individual is said to “tolerate” a dose of a compound if administration of that dose to that individual does not result in an unacceptable adverse event or an unacceptable combination of adverse events. One of skill in the art will appreciate that tolerance is a subjective measure and that what may be tolerable to one individual may not be tolerable to a different individual. For example, one individual may not be able to tolerate headache, whereas a second individual may find headache tolerable but is not able to tolerate vomiting, whereas for a third individual, either headache alone or vomiting alone is tolerable, but the individual is not able to tolerate the combination of headache and vomiting (even if the severity of each is less than when experienced alone).

ADVERSE EVENT: As used herein, an “adverse event” is an untoward medical occurrence that is associated with treatment with Compound 1 or a pharmaceutically acceptable salt, solvate or hydrate thereof. In one embodiment, an adverse event is selected from dizziness, headache, and nausea.

It is understood that when the phrase “pharmaceutically acceptable salts, solvates and hydrates” or the phrase “pharmaceutically acceptable salt, solvate or hydrate” is used when referring to Compound 1, it embraces pharmaceutically acceptable solvates and/or hydrates of Compound 1, pharmaceutically acceptable salts of Compound 1, as well as pharmaceutically acceptable solvates and/or hydrates of pharmaceutically acceptable salts of Compound 1. It is also understood that when the phrase “pharmaceutically acceptable solvates and hydrates” or the phrase “pharmaceutically acceptable solvate or hydrate” is used when referring to Compound 1 that are salts, it embraces pharmaceutically acceptable solvates and/or hydrates of such salts.

It will be apparent to those skilled in the art that the dosage forms described herein may comprise, as the active component, either Compound 1 or a pharmaceutically acceptable salt or as a solvate or hydrate thereof. Moreover, various hydrates and solvates of Compound 1 and their salts will find use as intermediates in the manufacture of pharmaceutical compositions. Typical procedures for making and identifying suitable hydrates and solvates, outside those mentioned herein, are well known to those in the art. See, e.g., pp. 202-209 of K. J. Guillory, “Generation of Polymorphs, Hydrates, Solvates, and Amorphous Solids,” in: Polymorphism in Pharmaceutical Solids, ed. Harry G. Britain, Vol. 95, Marcel Dekker, Inc., New York, 1999. Accordingly, one aspect of the present disclosure pertains to methods of prescribing and/or administering hydrates and solvates of Compound 1 and/or its pharmaceutical acceptable salts, that can be isolated and characterized by methods known in the art, such as, thermogravimetric analysis (TGA), TGA-mass spectroscopy, TGA-Infrared spectroscopy, powder X-ray diffraction (XRPD), Karl Fisher titration, high resolution X-ray diffraction, and the like.

As used herein, the term “greater than” is used interchangeably with the symbol > and the term less than is used interchangeably with the symbol <. Likewise, the term greater than or equal to is interchangeably with the symbol ≥, and the term less than or equal to is interchangeably with the symbol ≤.

When an integer is used in a method disclosed herein, the term “about” can be inserted before the integer.

Throughout this specification, unless the context requires otherwise, the word “comprise”, or variations such as “comprises,” or “comprising” will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention(s) described herein is susceptible to variations and modifications other than those specifically described. It is to be understood that the invention(s) includes all such variations and modifications. The invention(s) also includes all of the steps, features, compositions and compounds referred to or indicated in this specification, individually or collectively, and any and all combinations or any two or more of said steps or features unless specifically stated otherwise.

The present invention(s) is not to be limited in scope by the specific embodiments described herein, which are intended for the purpose of exemplification only. Functionally equivalent products, compositions and methods are clearly within the scope of the invention(s), as described herein. For example, the dosage amounts of Compound 1 or pharmaceutically acceptable salts, solvates or hydrates thereof disclosed herein can be replaced with dosage amounts for other salt or crystalline forms, formulations, and dosage regimens that exhibit bioequivalence to the specified amount of Compound 1 or pharmaceutically acceptable salts, solvates or hydrates thereof, including forms with 80-125% of the AUC and/or Cₘₐₓ as measured by methods disclosed in the FDA's Guidance for Industry for Bioavailability and Bioequivalence (Guidance for Industry: Bioavailability and Bioequivalence Studies for Orally Administered Drug Products—General Considerations. U.S. Department of Health and Human Services, Food and Drug Administration, Center for Drug Evaluation and Research (CDER), March 2003, Revision 1, www.fda.gov/cder/guidance/index.htm). For example, the dosage amounts of Compound 1 disclosed herein can be replaced with dosage amounts for other salt or crystalline forms, formulations, or dosage regimens that exhibit bioequivalence to that dosage amount of Compound 1.

It is appreciated that certain features of the invention(s), which are, for clarity, described in the context of separate embodiments, can also be provided in combination in a single embodiment. For example, a method that recites prescribing Compound 1 or a pharmaceutically acceptable salt, solvate or hydrate thereof and a separate method of the invention reciting administering Compound 1 or a pharmaceutically acceptable salt, solvate or hydrate thereof can be combined into a single method reciting prescribing and administering Compound 1 or a pharmaceutically acceptable salt, solvate or hydrate thereof.

Provided is a method of treating or preventing Raynaud's, comprising prescribing and/or administering to an individual in need thereof 3-methoxy-N-[3-(2-metyhylpyrazol-3-yl)-4-(2-morpholin-4-ylethoxy)phenyl]benzamide (Compound 1), or a pharmaceutically acceptable salt, hydrate, or solvate thereof.

Provided is a method of treating or preventing SSc-RP, comprising prescribing and/or administering to an individual in need thereof 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof.

In some embodiments, Raynaud's is Raynaud's syndrome. In some embodiments, Raynaud's is Raynaud's disease. In some embodiments, Raynaud's is Raynaud's phenomenon. In some embodiments, Raynaud's is primary Raynaud's syndrome. In some embodiments, Raynaud's is secondary Raynaud's syndrome. In some embodiments, Raynaud's is primary Raynaud's disease. In some embodiments, Raynaud's is secondary Raynaud's disease. In some embodiments, Raynaud's is primary Raynaud's phenomenon. In some embodiments, Raynaud's is secondary Raynaud's phenomenon. In some embodiments, Raynaud's is Raynaud's disease secondary to systemic sclerosis. In some embodiments, Raynaud's is Raynaud's disease secondary to another autoimmune disease. In some embodiments, Raynaud's is Raynaud's syndrome secondary to systemic sclerosis. In some embodiments, Raynaud's is Raynaud's syndrome secondary to another autoimmune disease. In some embodiments, Raynaud's is Raynaud's phenomenon secondary to an autoimmune disease. In some embodiments, Raynaud's is progressive systemic sclerosis (PSS).

In some embodiments, the individual has at least one digital ulcer.

In some embodiments, the individual has digital gangrene.

In some embodiments, the individual has systemic sclerosis.

In some embodiments, the individual has Raynaud's secondary to a condition selected from: systemic sclerosis, rheumatoid arthritis, lupus, vasculitis, atherosclerosis, cryoglobulinemia, hypothyroidism, injury, and drug reaction.

In some embodiments, the individual has Raynaud's secondary to systemic sclerosis.

In some embodiments, the individual has systemic sclerosis and at least one digital ulcer.

In some embodiments, treating comprises at least one of the following: a reduction in the frequency of attacks, a reduction in the duration of attacks, a reduction in the severity of attacks, a reduction in ischemic tissue injury, and a reduction in digital ulceration.

In some embodiments, treating comprises a reduction in the frequency, duration, and/or severity of attacks.

In some embodiments, preventing comprises at least one of the following: the prevention of attacks, the prevention of ischemic tissue injury, and the prevention of digital ulceration.

In some embodiments, about 30, 35, 40, 45, 50, 55, 60, 65, 70, 75, 80, 85, 90, 95, 100, 105, 110, 115, 120, 125, 130, 135, 140, 145, 150, 155, or 160 mg of the Compound 1 or pharmaceutically acceptable salt, hydrate, or solvate thereof is administered per day.

In some embodiments, about 10, 15, 20, 25, 30, 35, 40, 45, 50, 55, 60, 65, 70, 75, 80, 85, 90, 95, or 100 mg of the Compound 1 or pharmaceutically acceptable salt, hydrate, or solvate thereof is administered.

In some embodiments, Compound 1 or pharmaceutically acceptable salt, hydrate, or solvate thereof is administered three times daily (TID).

In some embodiments, Raynaud's is determined using a patient reported outcome (PRO).

In some embodiments, Compound 1 or pharmaceutically acceptable salt, hydrate, or solvate thereof is administered during the winter.

In some embodiments, the method further comprises instructing the individual to minimize exposure to ultraviolet light. In some embodiments, the method further comprises instructing the individual to wear protective clothing, protective eyewear, and apply sunscreen to exposed parts of the body.

In some embodiments, the methods of treatment provided herein are maintenance treatments for an individual who has previously received or is currently receiving another therapy for Raynaud's. In some embodiments, the individual with Raynaud's has been resistant to previous Raynaud's therapy. For example, in some embodiments, the individual with Raynaud's has been resistant to Ca2+ blocker, anti-platelet, topical nitroglycerin, ACE inhibitor or ARB, alpha blocker, SSRI or other anti-anxiolytic, PDE5, ETRA, or IV prostacyclin analogue therapy. In some embodiments, the individual with Raynaud's has been resistant to vasodilatory therapy. In some embodiments, the individual with Raynaud's has been resistant to intravenous prostanoid therapy. In some embodiments, the individual with Raynaud's has been resistant to epoprostenol, iloprost, bosentan, tadalafil, nifedipine, nicardipine, or quinapril therapy.

In some embodiments, Compound 1, or a pharmaceutically acceptable salt, hydrate, or solvate thereof, is administered in an amount equivalent to between about 10 mg and about 250 mg of Compound 1 free base.

In some embodiments, Compound 1, or a pharmaceutically acceptable salt, hydrate, or solvate thereof, is administered in an amount equivalent to between about 20 mg and about 250 mg of Compound 1 free base.

In some embodiments, Compound 1, or a pharmaceutically acceptable salt, hydrate, or solvate thereof, is administered in an amount equivalent to between about 10 mg and about 200 mg of Compound 1 free base.

In some embodiments, Compound 1, or a pharmaceutically acceptable salt, hydrate, or solvate thereof, is administered in an amount equivalent to between about 10 mg and about 80 mg of Compound 1 free base.

In some embodiments, Compound 1, or a pharmaceutically acceptable salt, hydrate, or solvate thereof, is administered in an amount equivalent to between about 10 mg and about 60 mg of Compound 1 free base.

In some embodiments, Compound 1, or a pharmaceutically acceptable salt, hydrate, or solvate thereof, is administered in an amount equivalent to about 10 mg of Compound 1 free base. In some embodiments, Compound 1, or a pharmaceutically acceptable salt, hydrate, or solvate thereof, is administered in an amount equivalent to about 15 mg of Compound 1 free base. In some embodiments, Compound 1, or a pharmaceutically acceptable salt, hydrate, or solvate thereof, is administered in an amount equivalent to about 20 mg of Compound 1 free base. In some embodiments, Compound 1, or a pharmaceutically acceptable salt, hydrate, or solvate thereof, is administered in an amount equivalent to about 25 mg of Compound 1 free base. In some embodiments, the Compound 1, or a pharmaceutically acceptable salt, hydrate, or solvate thereof, is administered in an amount equivalent to about 30 mg of Compound 1 free base. In some embodiments, Compound 1, or a pharmaceutically acceptable salt, hydrate, or solvate thereof, is administered in an amount equivalent to about 40 mg of Compound 1 free base. In some embodiments, the Compound 1, or a pharmaceutically acceptable salt, hydrate, or solvate thereof, is administered in an amount equivalent to about 50 mg of Compound 1 free base. In some embodiments, Compound 1, or a pharmaceutically acceptable salt, hydrate, or solvate thereof, is administered in an amount equivalent to about 60 mg of Compound 1 free base. In some embodiments, the Compound 1, or a pharmaceutically acceptable salt, hydrate, or solvate thereof, is administered in an amount equivalent to about 70 mg of Compound 1 free base. In some embodiments, Compound 1, or a pharmaceutically acceptable salt, hydrate, or solvate thereof, is administered in an amount equivalent to about 75 mg of Compound 1 free base. In some embodiments, Compound 1, or a pharmaceutically acceptable salt, hydrate, or solvate thereof, is administered in an amount equivalent to about 80 mg of Compound 1 free base. In some embodiments, the Compound 1, or a pharmaceutically acceptable salt, hydrate, or solvate thereof, is administered in an amount equivalent to about 90 mg of Compound 1 free base. In some embodiments, the Compound 1, or a pharmaceutically acceptable salt, hydrate, or solvate thereof, is administered in an amount equivalent to about 100 mg of Compound 1 free base. In some embodiments, the Compound 1, or a pharmaceutically acceptable salt, hydrate, or solvate thereof, is administered in an amount equivalent to about 110 mg of Compound 1 free base. In some embodiments, the Compound 1, or a pharmaceutically acceptable salt, hydrate, or solvate thereof, is administered in an amount equivalent to about 120 mg of Compound 1 free base. In some embodiments, the Compound 1, or a pharmaceutically acceptable salt, hydrate, or solvate thereof, is administered in an amount equivalent to about 130 mg of Compound 1 free base. In some embodiments, the Compound 1, or a pharmaceutically acceptable salt, hydrate, or solvate thereof, is administered in an amount equivalent to about 140 mg of Compound 1 free base. In some embodiments, the Compound 1, or a pharmaceutically acceptable salt, hydrate, or solvate thereof, is administered in an amount equivalent to about 150 mg of Compound 1 free base. In some embodiments, the Compound 1, or a pharmaceutically acceptable salt, hydrate, or solvate thereof, is administered in an amount equivalent to about 160 mg of Compound 1 free base. In some embodiments, the Compound 1, or a pharmaceutically acceptable salt, hydrate, or solvate thereof, is administered in an amount equivalent to about 170 mg of Compound 1 free base. In some embodiments, the Compound 1, or a pharmaceutically acceptable salt, hydrate, or solvate thereof, is administered in an amount equivalent to about 180 mg of Compound 1 free base. In some embodiments, the Compound 1, or a pharmaceutically acceptable salt, hydrate, or solvate thereof, is administered in an amount equivalent to about 190 mg of Compound 1 free base. In some embodiments, the Compound 1, or a pharmaceutically acceptable salt, hydrate, or solvate thereof, is administered in an amount equivalent to about 200 mg of Compound 1 free base. In some embodiments, the Compound 1, or a pharmaceutically acceptable salt, hydrate, or solvate thereof, is administered in an amount equivalent to about 210 mg of Compound 1 free base. In some embodiments, the Compound 1, or a pharmaceutically acceptable salt, hydrate, or solvate thereof, is administered in an amount equivalent to about 220 mg of Compound 1 free base. In some embodiments, the Compound 1, or a pharmaceutically acceptable salt, hydrate, or solvate thereof, is administered in an amount equivalent to about 230 mg of Compound 1 free base. In some embodiments, the Compound 1, or a pharmaceutically acceptable salt, hydrate, or solvate thereof, is administered in an amount equivalent to about 240 mg of Compound 1 free base. In some embodiments, the Compound 1, or a pharmaceutically acceptable salt, hydrate, or solvate thereof, is administered in an amount equivalent to about 250 mg of Compound 1 free base.

In some embodiments, the Compound 1, or a pharmaceutically acceptable salt, hydrate, or solvate thereof, is administered once daily (QD).

In some embodiments, the Compound 1, or a pharmaceutically acceptable salt, hydrate, or solvate thereof, is administered twice daily (BID).

In some embodiments, the Compound 1, or a pharmaceutically acceptable salt, hydrate, or solvate thereof, is administered three times daily (TID).

In some embodiments, Compound 1, or a pharmaceutically acceptable salt, hydrate, or solvate thereof, is administered in an amount equivalent to between about 10 mg and about 80 mg of Compound 1 free base TID (for a total equivalent to between about 30 mg and about 240 mg of Compound 1 free base daily).

In some embodiments, Compound 1, or a pharmaceutically acceptable salt, hydrate, or solvate thereof, is administered in an amount equivalent to between about 10 mg and about 60 mg of Compound 1 free base TID (for a total equivalent to between about 30 mg and about 180 mg of Compound 1 free base daily).

In some embodiments, the Compound 1, or a pharmaceutically acceptable salt, hydrate, or solvate thereof, is administered orally.

In some embodiments, the oral formulation is administered for at least, or at least about, 1, 2, 3, 4, 5, 6, 7, 8, 9, 10, 11, or 12 months.

In some embodiments, the Compound 1, or a pharmaceutically acceptable salt, hydrate, or solvate thereof, is formulated as a capsule suitable for oral administration. In some embodiments, the Compound 1, or a pharmaceutically acceptable salt, hydrate, or solvate thereof, is formulated as a tablet suitable for oral administration.

In some embodiments, the Compound 1, or a pharmaceutically acceptable salt, hydrate, or solvate thereof, is a pharmaceutically acceptable salt of Compound 1, or a hydrate or solvate thereof.

In some embodiments, the Compound 1, or a pharmaceutically acceptable salt, hydrate, or solvate thereof, is a pharmaceutically acceptable salt of Compound 1.

In some embodiments, the Compound 1, or a pharmaceutically acceptable salt, hydrate, or solvate thereof, is a HCl salt of Compound 1. In some embodiments, the Compound 1, or a pharmaceutically acceptable salt, hydrate, or solvate thereof, is a hydrate of Compound 1. In some embodiments, the Compound 1, or a pharmaceutically acceptable salt, hydrate, or solvate thereof, is a solvate of Compound 1.

In some embodiments the Compound 1, or a pharmaceutically acceptable salt, hydrate, or solvate thereof is useful in the inhibition of platelet aggregation in Raynaud's phenomenon. In some embodiments, the Compound 1, or a pharmaceutically acceptable salt, hydrate, or solvate thereof is useful for the inhibition of vasoconstriction in Raynaud's phenomenon. In some embodiments, the Compound 1, or a pharmaceutically acceptable salt, hydrate, or solvate thereof is useful for the inhibition of platelet aggregation and vasoconstriction in Raynaud's phenomenon.

The compounds disclosed herein are useful in the treatment of systemic sclerosis and symptoms thereof. Systemic sclerosis can be, for example, CREST (calcinosis, Raynaud's, esophageal dysmotility, sclerodactyly, and telangiectasia) syndrome. Symptoms of systemic sclerosis include, for example, Raynaud's.

The compounds disclosed herein are useful in the treatment of Raynaud's and symptoms thereof. Symptoms of Raynaud's syndrome include, for example, digital ulceration and gangrene. The compounds disclosed herein are useful in the treatment of symptoms of Raynaud's syndrome.

In some embodiments, the treatment or prevention of Raynaud's is determined by the nature of Raynaud's attacks. In some embodiments, the treatment or prevention of Raynaud's is determined by the frequency of Raynaud's attacks. In some embodiments, the treatment or prevention of Raynaud's is determined by the duration of Raynaud's attacks. In some embodiments, the treatment or prevention of Raynaud's is determined by the severity of Raynaud's attacks. In some embodiments, the treatment or prevention of Raynaud's is determined by the impact of Raynaud's attacks.

In some embodiments, the frequency of Raynaud's attacks is measured per day. In some embodiments, the frequency of Raynaud's attacks is measured per week. In some embodiments, the frequency of Raynaud's attacks is measured per month. In some embodiments, the frequency of Raynaud's attacks is measured per year. In some embodiments, the frequency of Raynaud's attacks is measured as the time between attacks.

In some embodiments, the duration of Raynaud's attacks is measured in minutes. In some embodiments, the duration of Raynaud's attacks is measured in hours. In some embodiments, duration of Raynaud's attacks is measured in days.

In some embodiments, the change in frequency, duration, and/or severity of Raynaud's attacks is determined compared to a baseline. In some embodiments, the change from baseline is determined at week 4, 6, 8, 10, 12, 16, 20, 24, 28, 32, 36, 20, 44, 48, or 52.

In some embodiments, the treatment or prevention of Raynaud's is determined by the reduction in ulcer burden in secondary Raynaud's. In some embodiments, the treatment or prevention of Raynaud's is determined by the healing of a digital ulcer. In some embodiments, the treatment or prevention of Raynaud's is determined by the healing of more than one digital ulcer. In some embodiments, the treatment or prevention of Raynaud's is determined by the healing of all digital ulcers. In some embodiments, the treatment or prevention of Raynaud's is determined by the prevention of new active digital ulcers. In some embodiments, the treatment or prevention of Raynaud's is determined by the time to heal one or more digital ulcers.

In some embodiments, the treatment or prevention of Raynaud's is determined by a visual analogue scale (VAS). In some embodiments, the treatment or prevention of Raynaud's is determined by a Raynaud Severity Visual Analog Score. In some embodiments, the treatment or prevention of Raynaud's is determined by a Raynaud's Condition Score Visual Analog Score. In some embodiments, the treatment or prevention of Raynaud's is determined by digital blood pressure. In some embodiments, the treatment or prevention of Raynaud's is determined by capillary diameter. In some embodiments, the treatment or prevention of Raynaud's is determined by the reduction in a hand disability score.

In some embodiments, the treatment or prevention of Raynaud's is determined by a patient's assessment. In some embodiments, the treatment or prevention of Raynaud's is determined by a physician's assessment.

In some embodiments, the treatment or prevention of Raynaud's is determined by a patient reported outcome (PRO). In some embodiments, the PRO is a Raynaud's Condition Score (RCS). In some embodiments, the PRO is a Likert scale. In some embodiments, the PRO is an 10-point Likert scale. In some embodiments, the PRO is an 11-point Likert scale. In some embodiments, the PRO is a daily patient assessment. In some embodiments, the PRO is an individual's assessment of pain during a Raynaud's attack. In some embodiments, the PRO is an individual's assessment of numbness during a Raynaud's attack. In some embodiments, the PRO is an individual's assessment of tingling during a Raynaud's attack. In some embodiments, the PRO is an individual's global assessment of the severity of Raynaud's. In some embodiments, the PRO is an individual's assessment of quality of life (QOL) with Raynaud's.

In some embodiments, the treatment or prevention of Raynaud's is the treatment or prevention of a measurement described herein. For example, in some embodiments, the treatment or prevention of Raynaud's is an improvement in a PRO.

In some embodiments, the methods described herein are useful for the prevention or treatment of critical ischemia in SSc-RP. In some embodiments, critical ischemia is the presence of ischemic symptoms at rest. In some embodiments, the methods described herein are useful for the prevention or treatment of severe ischemia in SSc-RP. In some embodiments, severe ischemia is the requirement for intravenous vasodilator therapy, surgical debridement and/or amputation.

In some embodiments, the methods described herein are useful for the prevention or treatment of finger discoloration. In some embodiments, the methods described herein are useful for the prevention or treatment of one or more digital ulcers. In some embodiments, the methods described herein are useful for the prevention or treatment of gangrene in SSc-RP. In some embodiments, the methods described herein are useful for the prevention of amputation. In some embodiments, the methods described herein are useful for the prevention of amputation of one or more fingers.

In some embodiments, the methods described herein are useful for the prevention or treatment of vasculopathy in SSc-RP. In some embodiments, the methods described herein are useful for the prevention or treatment of endothelial dysfunction in SSc-RP. In some embodiments, the methods described herein are useful for the prevention or treatment of tissue fibrosis in SSc-RP.

In some embodiments, the methods described herein are useful for improving digital blood flow. In some embodiments, the methods described herein are useful for improving digital blood pressure. In some embodiments, the methods described herein are useful for decreasing digital capillary dysfunction. In some embodiments, the methods described herein are useful for decreasing endothelial dysfunction. In some embodiments, the methods described herein are useful for the prevention or treatment of reduced blood flow following exposure to cold. In some embodiments, the methods described herein are useful for improving the recovery of blood flow following exposure to cold.

In some embodiments, the compounds described herein are administered seasonally. In some embodiments, the compounds described herein are administered during winter. In some embodiments, the compounds described herein are administered when an individual is exposed to temperatures less than, or less than about, 60° F., 55° F., 50° F., 45° F., 40° F., 35° F., or 32° F. In some embodiments, the compounds described herein are administered upon the detection of a symptom described herein. In some embodiments, a blood test is used to diagnose secondary Raynaud's phenomenon.

In some embodiments, the method further comprises monitoring of bleeding-related adverse events. In some embodiments, the assessment will be categorized using the BARC definition for bleeding as set forth in Mehran et al. (2011) Circulation 123(23)2736-2747, wherein:
Type 0 is defined as no bleeding;
Type 1 is defined as bleeding that is not actionable and does not cause the patient to seek unscheduled performance of studies, hospitalization, or treatment by a healthcare professional; may include episodes leading to self-discontinuation of medical therapy by the patient without consulting a healthcare professional;
Type 2 is defined as any overt, actionable sign of hemorrhage (i.e., more bleeding than would be expected for a clinical circumstance, including bleeding found by imaging alone) that does not fit the criteria for Type 3, 4, or 5 but does meet at least one of the following criteria:
   Requiring nonsurgical, medical intervention by a healthcare professional.
   Leading to hospitalization or increased level of care.
   Prompting evaluation;
Type 3 is defined as:
   Overt bleeding plus hemoglobin drop of 3 to <5 g/dLa (provided hemoglobin drop is related to bleed). Any transfusion with overt bleeding,
   Overt bleeding plus hemoglobin drop ≥5 g/dLa (provided hemoglobin drop is related to bleed). Cardiac tamponade bleeding requiring surgical intervention for control (excluding dental/nasal/skin/hemorrhoid). Bleeding requiring intravenous vasoactive agents; or
   Intracranial hemorrhage (does not include microbleeds or hemorrhagic transformation does include intraspinal). Subcategories confirmed by autopsy or imaging or lumbar puncture. Intraocular bleed compromising vision;
Type 4 is defined as CABG-related bleeding. Perioperative intracranial bleeding within 48 hours. Reoperation after closure of sternotomy for the purpose of controlling bleeding. Transfusion of ≥5 U whole blood or packed red blood cells within a 48-hour period. Chest tube output ≥2 L within a 24-hour period; and
Type 5 is defined as fatal bleeding.

Also provided is a method of identifying a subject who is likely to be responsive to a treatment with Compound 1, or a pharmaceutically acceptable salt, hydrate, or solvate thereof, comprising:
administering Compound 1, or a pharmaceutically acceptable salt, hydrate, or solvate thereof, to the subject;
obtaining a sample from the subject;
determining the level of a biomarker in the sample; and
diagnosing the subject as being likely to be responsive to the treatment compound if the level of the biomarker in the sample is different from a reference level of the biomarker.

In some embodiments, the sample is a blood sample. In some embodiments, the sample is a tissue sample.

In some embodiments, the biomarker is plasma serotonin concentration, markers of vascular injury and endothelial dysfunction (endothelin-1, factor VIII activity, intracellular adhesion molecule 1 [ICAM-1], thromboxane B2, tissue-type plasminogen activator antigen [t-PA], plasminogen activator inhibitor-1 [PAI-1] activity, PAI-1 antigen, vascular adhesion molecule 1 (VCAM-1), vascular endothelial growth factor (VEGF), and von Willebrand factor [VWF]).

In some embodiments, the level of the biomarker in the sample is higher than the reference level of the biomarker. In some embodiments, the level of the biomarker in the sample is lower than the reference level of the biomarker.

In some embodiments, the level of the biomarker is measured by determining the protein level of the biomarker. In some embodiments, the level of the biomarker is measured by determining the mRNA level of the biomarker. In some embodiments, the level of the biomarker is measured by determining the cDNA level of the biomarker. In some embodiments, the biomarker is determined by DNA sequencing. In some embodiments, the biomarker is determined by RNA-sequencing (RNA-seq).

Also provided herein is the use of Compound 1, or a pharmaceutically acceptable salt, hydrate, or solvate thereof, as described herein, in the manufacture of a medicament for the treatment of Raynaud's as described herein.

Also provided herein is Compound 1, or a pharmaceutically acceptable salt, hydrate, or solvate thereof, as described herein, for use in a method of treatment of Raynaud's as described herein.

Also provided herein is Compound 1, or a pharmaceutically acceptable salt, hydrate, or solvate thereof, as described herein, for the treatment of Raynaud's as described herein.

Also provided herein is a kit comprising a titration package as disclosed herein and instructions indicating that the medication is to be administered to an individual in need of treatment for Raynaud's.

Conventional excipients, such as binding agents, fillers, acceptable wetting agents, tabletting lubricants and disintegrants can be used in tablets and capsules for oral administration. Liquid preparations for oral administration can be in the form of solutions, emulsions, aqueous or oily suspensions and syrups. Alternatively, oral preparations can be in the form of dry powder that can be reconstituted with water or another suitable liquid vehicle before use. Additional additives such as suspending or emulsifying agents, non-aqueous vehicles (including edible oils), preservatives and flavorings and colorants can be added to the liquid preparations.

Parenteral dosage forms can be prepared by dissolving the compound in a suitable liquid vehicle and filter sterilizing the solution before filling and sealing an appropriate vial or ampule. These are just a few examples of the many appropriate methods well known in the art for preparing dosage forms. Suitable pharmaceutically-acceptable carriers, outside those mentioned herein, are known in the art. See, e.g., Remington, The Science and Practice of Pharmacy, 20^{th} Edition, 2000, Lippincott Williams & Wilkins, (Editors: Gennaro et al.).

The compounds provided herein can be administrated in a wide variety of oral and parenteral dosage forms. It will be obvious to those skilled in the art that the dosage forms may comprise, as the active component, either a compound provided herein or a pharmaceutically acceptable salt, solvate or hydrate of a compound provided herein.

In some embodiments, Compound 1 is in a pharmaceutical formulation or further comprises a pharmaceutically acceptable carrier. Pharmaceutical formulations include those suitable for oral, rectal, nasal, topical (including buccal and sub-lingual), vaginal or parenteral (including intramuscular, subcutaneous and intravenous) administration or in a form suitable for administration by inhalation, insufflation, or by transdermal patch. The compounds provided herein, together with a conventional adjuvant, carrier, or diluent, can thus be placed into the form of pharmaceutical formulations and unit dosages thereof and in such form may be employed as solids, such as tablets or filled capsules, or liquids, such as solutions, suspensions, emulsions, elixirs, gels or capsules filled with the same, all for oral use, in the form of suppositories for rectal administration, or in the form of sterile injectable solutions for parenteral (including subcutaneous) use. The active ingredient may also be administered by injection as a composition wherein, for example, saline, dextrose or water may be used as a suitable pharmaceutically acceptable carrier. Such pharmaceutical compositions and unit dosage forms thereof can comprise conventional ingredients in conventional proportions, with or without additional active compounds or principles and such unit dosage forms may contain any suitable effective amount of the active ingredient commensurate with the intended daily dosage range to be employed. In some embodiments, the capsule is a gelatin capsule. In some embodiments, the capsule is a hard gelatin capsule.

For oral administration, the pharmaceutical composition can be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate.

In some embodiments, the oral formulation of Compound 1 or pharmaceutically acceptable salt, hydrate, or solvate thereof, is an immediate-release dosage form comprising Compound 1, or a pharmaceutically acceptable salt, hydrate, or solvate thereof.

In some embodiments, the Compound 1, or a pharmaceutically acceptable salt, hydrate, or solvate thereof, is formulated as a capsule suitable for oral administration. In some embodiments, the Compound 1, or a pharmaceutically acceptable salt, hydrate, or solvate thereof, is formulated as a tablet suitable for oral administration.

In some embodiments, the Compound 1, or a pharmaceutically acceptable salt, hydrate, or solvate thereof, is co-administered with at least one anti-platelet drug. In some embodiments, the Compound 1, or a pharmaceutically acceptable salt, hydrate, or solvate thereof, is co-administered with dual anti-platelet therapy (DAPT). In some embodiments, the individual is already being administered at least one anti-platelet drug prior to the administration of Compound 1, or a pharmaceutically acceptable salt, hydrate, or solvate thereof as described herein. In some embodiments, the individual is administered a first dose of at least one anti-platelet drug prior to PCI. In some embodiments, the individual is administered a loading dose of at least one anti-platelet drug prior to PCI. In some embodiments, the anti-platelet drug is aspirin. In some embodiments, the anti-platelet drug is a P2Y12 inhibitor. In some embodiments, the P2Y12 inhibitor is an oral P2Y12 inhibitor. In some embodiments, the P2Y12 inhibitor is selected from clopidogrel, ticlopidine, ticagrelor, prasugrel, and cangrelor. In some embodiments, the DAPT is aspirin and clopidogrel. In some embodiments, the Compound 1, or a pharmaceutically acceptable salt, hydrate, or solvate thereof, is co-administered with clopidogrel, but not prasugrel or ticagrelor.

In some embodiments, an individual is co-administered a vasodilating agent. In some embodiments, an individual is co-administered a calcium channel blocker (CCB), a phosphodiesterase (PDE) 5 inhibitor, an angiotensin II receptor blocker (ARB), a topical nitrate, intravenous (IV) prostanoid and/or an endothelin-1 receptor antagonist.

In some embodiments, an individual is not co-administered an inhibitor or inducer of CYP3A4, CYP3A5, and/or P-glycoprotein with Compound 1, or a pharmaceutically acceptable salt, hydrate, or solvate thereof. In some embodiments, the individual is already being administered at least one inhibitor or inducer of CYP3A4, CYP3A5, and/or P-glycoprotein prior to the administration of Compound 1, or a pharmaceutically acceptable salt, hydrate, or solvate thereof as described herein. In some embodiments, the administration of an inhibitor or inducer of CYP3A4, CYP3A5, and/or P-glycoprotein is discontinued when an individual is administered Compound 1, or a pharmaceutically acceptable salt, hydrate, or solvate thereof. In some embodiments, an individual is co-administered a lower dose of an inhibitor or inducer of CYP3A4, CYP3A5, and/or P-glycoprotein when co-administered with Compound 1, or a pharmaceutically acceptable salt, hydrate, or solvate thereof. In some embodiments, an individual is administered a lower dose of Compound 1, or a pharmaceutically acceptable salt, hydrate, or solvate thereof when co-administered with an inhibitor or inducer of CYP3A4, CYP3A5, and/or P-glycoprotein. In some embodiments, an individual is co-administered a lower dose of an inhibitor or inducer of CYP3A4, CYP3A5, and/or P-glycoprotein and a lower dose of Compound 1, or a pharmaceutically acceptable salt, hydrate, or solvate thereof. In some embodiments, the inhibitor or inducer of CYP3A4, CYP3A5, and/or P-glycoprotein is a strong inhibitor or inducer of CYP3A4, CYP3A5, and/or P-glycoprotein. In some embodiments, the inhibitor or inducer of CYP3A4, CYP3A5, and/or P-glycoprotein is selected from ketoconazole, itraconazole, clarithromycin, erythromycin, rifampicin, and verapamil. In some embodiments, the inhibitor or inducer of CYP3A4, CYP3A5, and/or P-glycoprotein is an inhibitor or inducer of CYP3A4. In some embodiments, the inhibitor or inducer of CYP3A4, CYP3A5, and/or P-glycoprotein is an inhibitor or inducer of CYP3A5. In some embodiments, the inhibitor or inducer of CYP3A4, CYP3A5, and/or P-glycoprotein is an inhibitor or inducer of P-glycoprotein. In some embodiments, the individual is co-administered intra-arterial verapamil for radial artery PCI.

Some embodiments include a method of producing a pharmaceutical composition for fixed dose combination therapy comprising admixing at least one compound according to any of the compound embodiments disclosed herein, together with at least one known pharmaceutical agent as described herein and a pharmaceutically acceptable carrier.

Further embodiments include the embodiments disclosed in the following Examples, which are not to be construed as limiting in any way.

### Example 1

A study was conducted to assess the effects of intra-arterial dosing of Compound 1 and ketanserin on body temperature, peripheral cooling and peripheral blood flow in naïve anesthetized male New Zealand White rabbits.

| | | | Test | | | | |
|---|---|---|---|---|---|---|---|
| | | | Compound | Dose | Dose | Infusion | |
| | Test | Dose | Dosage | Conc. | Volume | Time | # of Male |
| Group | Compound | Number | (mg/kg) | (mg/mL) | (mL/kg) | (min) | Rabbits |
| 1 | vehicle¹ | 1 | 0 | 0 | 0.5 | 15 | 4 |
| | | 2 | 0 | 0 | 1.0 | 30 | |
| 2 | ketanserin | 1 | 0.05 | 0.1 | 0.5 | 15 | 4 |
| | | 2 | 0.1 | 0.1 | 1.0 | 30 | |
| 3 | Compound 1 | 1 | 0.05 | 0.1 | 0.5 | 15 | 4 |
| | | 2 | 0.1 | 0.1 | 1.0 | 30 | |
| 4² | vehicle¹ | 1 | 0 | 0 | 0.5 | 15 | 3 |
| | | 2 | 0 | 0 | 1.0 | 45 | |
| 5² | ketanserin | 1 | 0.05 | 0.1 | 0.5 | 15 | 3 |
| | | 2 | 0.1 | 0.1 | 1.0 | 45 | |
| 6² | Compound 1 | 1 | 0.05 | 0.1 | 0.5 | 15 | 3 |
| | | 2 | 0.1 | 0.1 | 1.0 | 45 | |

¹Vehicle was 0.9% NaCl in water, U.S.P. (saline).
²Following 30 minutes of dosing with Dose 2, each rabbit's left foot was removed to room temperature.
They were then monitored and dosed for an additional 15 minutes.
Dose 1 was delivered over 15 minutes with left foot in 40° C. water bath.
Dose 2 delivered over 30 minutes with left foot in 15° C. water bath.

Formulations of each test compound were prepared by adding saline and vortexing and sonicating until a clear solution was obtained.

Rabbits were sedated with ketamine/xylazine (25/5 mg/kg, subcutaneous) and then intubated and anesthetized with 1-2% isoflurane driven by 100% oxygen (1-2 L/min). Animals were mechanically ventilated and normal body temperature (38-40° C.) was maintained using an external source of heat (e.g., circulating water blanket, heated surgery table). The femoral incision site was infiltrated with 1 mg/kg ID bupivacaine. A catheter for dosing was placed via cutdown in the left femoral artery.

Temperature thermistors were placed on the skin of the dorsal surfaces of the left hind foot (LHFT) and right hind foot (RHFT) to assess peripheral temperature and into the rectum to assess core body temperature. Temperature values were reported in 1 minute averages from 5 minutes prior to initiation of Dose 1 administration (values averaged and used as baseline value) through the completion of Dose 2 administration.

A Transonics Systems, Inc. (Ithaca, NY) doppler flow probe was affixed to the dorsal surface of the left hind foot to assess peripheral blood flow. Peripheral blood flow (PBF) was reported in 1 minute averages from 5 minutes prior to initiation of Dose 1 administration (values averaged and used as baseline value) through the completion of Dose 2 administration.

Via surface leads, electrocardiography (ECG) was recorded.

At the start of Dose 1 administration, each rabbit's left hind foot was placed in a water bath set to maintain approximately 40° C. Immediately after Dose 1 administration, Dose 2 administration commenced. Each rabbit's left hind foot was placed in a water bath set to maintain approximately 15° C. at the start of Dose 2 administration. At 30 minutes post dose start of Dose 2, in Groups 4-6 only, each rabbit's left hind foot was removed to room temperature, monitored and dosed for an additional 15 minutes.

At the conclusion of Dose 2 administration, blood was sampled from the right and left femoral vein. The blood samples were collected in a collection tube containing K₂EDTA anticoagulant and 0.5 mM PGE1 (0.17725 mg/mL in ethanol) and 0.5 mM citalopram (0.20265 mg/mL in water) at a 1:500 ratio to blood volume (e.g., 0.5 mL of blood will contain 0.001 mL of PGE1 and 0.001 mL of citalopram). The sample was stored at room temperature for at least 30 minutes until spun in an ambient temperature centrifuge at approximately 850 g for 10 minutes. Approximately ⅔ of the volume of plasma was transferred. This was then spun at approximately 4500-5000 g for 5-10 minutes.

At the conclusion of Dose 2 administration, an additional 0.5 mL sample was collected from a systemic blood vessel into a collection tube containing K₂EDTA anticoagulant. The sample was spun in an ambient temperature centrifuge for approximately 10 minutes.

Average percent change of each temperature and blood flow between baseline and each minute of dose administration was determined relative to time-matched vehicle control data.

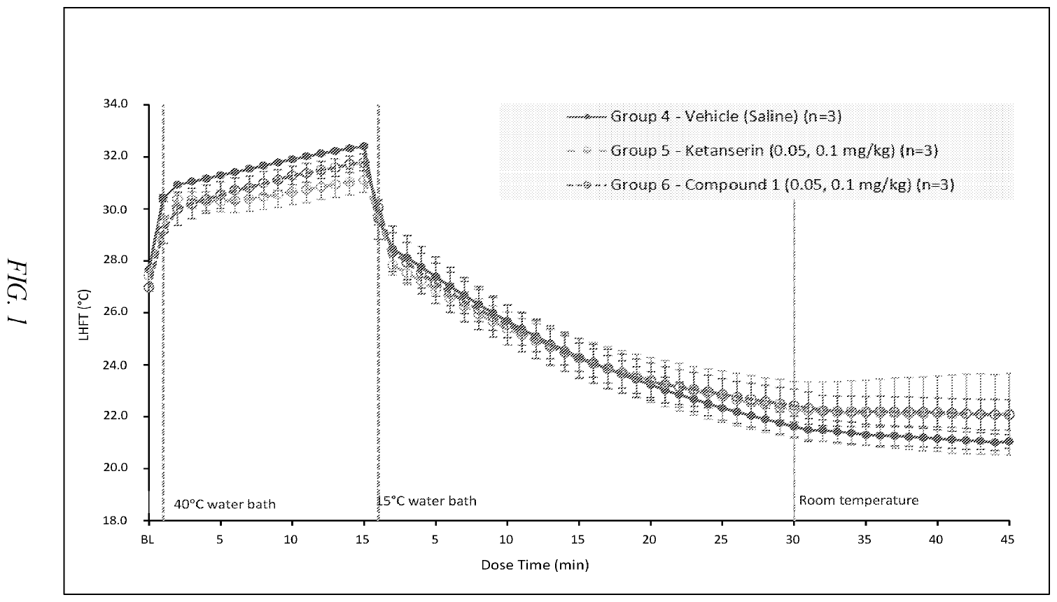

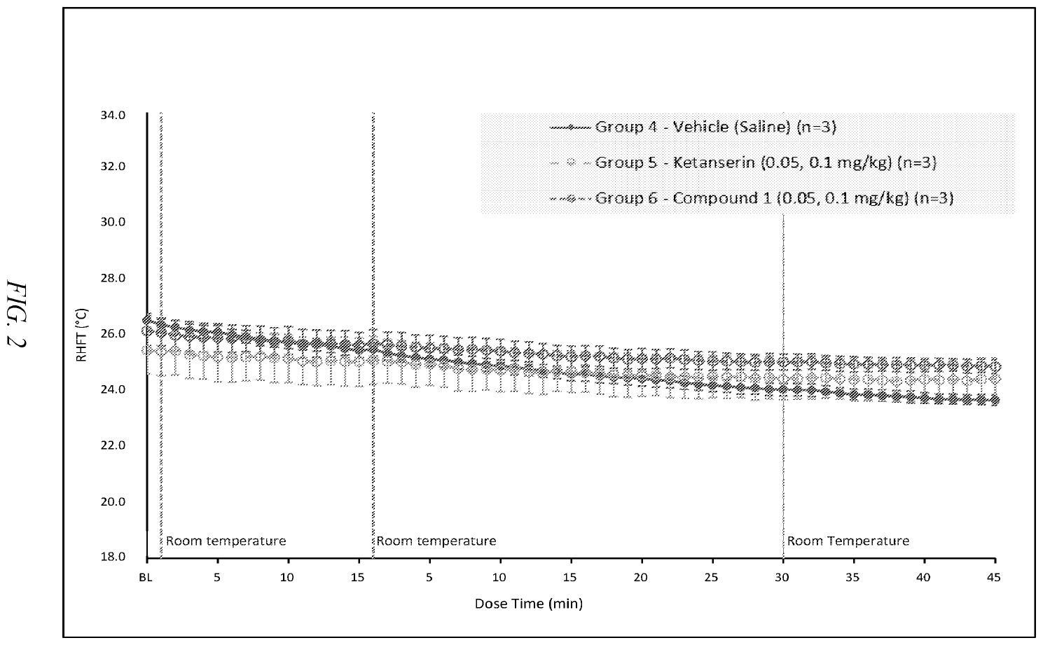

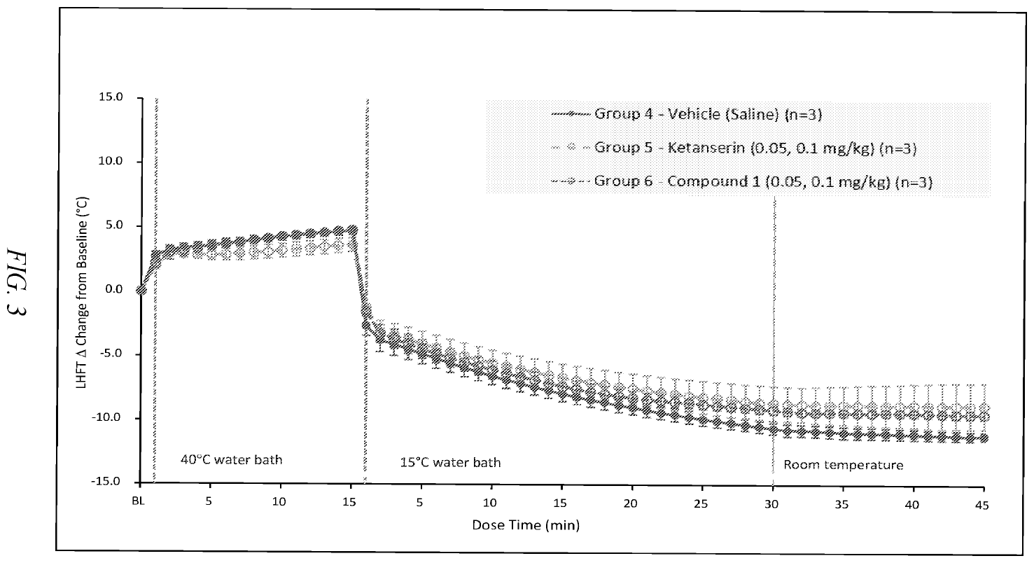

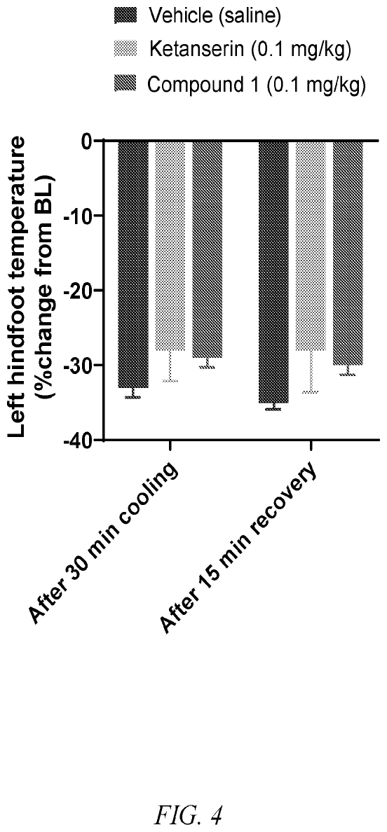

Compound 1 and ketanserin partially reduced cold-induced vasoconstriction in the rabbit foot (FIGS. 1-4). FIG. 1 shows left hind foot temperature in Groups 4-6 when introduced to 40° C., 15° C., and room temperature water bath. FIG. 2 shows right hind foot temperature in Groups 4-6 when introduced to room temperature water. FIG. 3 shows the change from baseline in left hind foot temperature in Groups 4-6 when introduced to 40° C., 15° C., and room temperature water bath. FIG. 4 shows the change from baseline in left hind foot temperature in Groups 4-6 at 30 minutes of cooling and 15 minutes of recovery.

### Example 2—Oral Formulation

An example of an oral formulation for Compound 1 is provided in Table 1:

TABLE 1
Immediate-Release Capsule

| | mg/capsule |
|---|---|
| Compound 1 HCl | 21.67 |
| Hard gelatin capsule | 61.00Error! Reference source not found. |
| Total | 82.67Error! Reference source not found. |

Error! Reference source not found.
approximate weight based on a typical empty capsule weight.

### Example 3—Clinical Trial 1

A phase 1, randomized, double-blind, placebo-controlled, single-dose escalation study was conducted to assess the tolerability, PK, and PD of Compound 1 doses of 1 to 320 mg administered orally as a single dose to a total of 91 healthy subjects.

Compound 1 was rapidly absorbed at all doses, and plasma exposure measures increased in a greater than dose proportional manner. The active metabolites M1 and M2 had longer half-lives than the parent compound, and as Compound 1 dose was escalated, Compound 1 half-life increased, exposure increased, and metabolite-to-parent ratios decreased.

PD assessment consisted of measuring pre- and post-dose platelet aggregation using an in vitro assay with ADP with and without 5-HT. The degree of thrombin receptor activating peptide (TRAP)-induced platelet aggregation inhibition between groups was similar. However, Compound 1 inhibited 5-HT-mediated amplification of platelet aggregation at doses above 1 mg.

5-HT-mediated platelet aggregation data from Compound 1 doses of 5 to 320 mg show a maximal inhibition at 1 to 2 hours and suggest a trend toward longer duration of inhibition with higher doses, although a high degree of variability confounded interpretation.

### Example 4—Clinical Trial 2

A phase 1, randomized, double-blind, placebo-controlled, multiple-ascending dose-escalation study was conducted to assess the safety, tolerability, PD, and steady state PK of Compound 1 from 5 to 80 mg administered orally TID for 7 days in a total of 50 healthy subjects.

Compound 1 was rapidly absorbed at all doses, and Day 1 AUC and Cmax values at equivalent doses were similar to those for the study in Example 3. Exposure as measured by AUC was dose proportional at 5 and 10 mg, but greater than dose proportional at 40, 60, and 80 mg on Day 1 and Day 7. The Cmax and AUC accumulation ratio values, were less than 2 for all doses when comparing Day 1 and Day 7, indicating no accumulation after TID dosing for 7 days. The half-life was similar at all doses and days.

The active metabolite M1 and M2 exposures increased and metabolite to parent ratios decreased with the increasing Compound 1 doses. The accumulation ratios as measured by AUCtau values were approximately 2 at 60 and 80 mg for M1 and M2, suggesting a 2-fold accumulation of these metabolites after 7 days of dosing TID.

Administration of Compound 1 inhibited ex vivo 5-HT-mediated amplification of platelet aggregation. Inhibition was essentially maximal following dosing on Days 1, 4, and 7 at both the 60 mg and 80 mg doses.

### Example 5—Clinical Trial 3

A phase 2, randomized, double-blind, placebo-controlled, crossover study will be conducted to assess the effect of oral Compound 1 on digital blood flow in subjects with Raynaud's phenomenon secondary to systemic sclerosis (SSc-RP). Raynaud's phenomenon is defined as a history of digital cold sensitivity associated with color changes of cyanosis and pallor, with on average at least 5 attacks per week or 2 attacks per day during the winter period secondary to systemic sclerosis based on either (1) a diagnosis of SSc using the American College of Rheumatology/European League Against Rheumatism and/or LeRoy and Medsger classification as set forth in LeRoy et al. (2001) J. Rheumatol. 28:1573-1576 or van den Hoogen et al. (2013) Arthritis Rheum 65:2737-2747 or (2) a diagnosis of very early SSc using the Very Early Diagnosis of Systemic Sclerosis (VEDOSS) criteria as set forth in Avouac et al. (2011) Ann. Rheum. Dis. 70(3):476-481.

Subjects will receive a single oral dose of study treatment on Day 1, Day 8, and Day 15 in a randomized 3-period crossover design. The oral formulation of Compound 1 contains active pharmaceutical ingredient as the HCl salt. In Part A of the study, each subject will receive a single administration of each dose strength. Selection of doses for Part B of the study will be based on the efficacy and safety results from Part A.

In Part A, subjects will be equally randomized to one of three crossover treatment sequences in a double-blinded manner. Each subject will participate in three treatment visits, which will each be separated by a washout period of at least 72 hours and up to 7 days. During each treatment visit, subjects will receive a single dose of study treatment (placebo, 60 mg Compound 1 free base, or 120 mg Compound 1 free base) according to their crossover treatment sequence assignment. On Day 1, subjects will be admitted to the clinic for treatment visit 1. Subjects will be instructed to consume a breakfast of approximately 400 to 500 calories with a fat content of less than 25% (11 to 14 g) at least 2 hours prior to treatment.

Following acclimatization, subjects will undergo pre-dose blood draws and assessments of temperature with infrared (IR) thermography or digital blood flow with laser speckle contrast imaging (LSCI). IR thermography is an indirect method for evaluation of blood flow based on imaging skin temperature, while LSCI is based on differences in the speckle pattern (occurring when laser light illuminates a tissue) due to movement of blood cells. Imaging software for IR thermography and LSCI provide quantitative measures of blood flow within predefined regions of interest (ROIs) as mean temperature (° C.) and arbitrary perfusion units (pu), respectively.

Upon study treatment administration, subjects will undergo re-acclimatization for at least 20 minutes. Blood samples for pharmacokinetic (PK) and plasma serotonin concentration will be collected 45 (plus or minus 5) minutes after dosing. Digital blood flow assessments will be performed again prior to cold challenge for 5 minutes at room temperature. A cold challenge will be conducted by immersing the hands in a temperature-controlled water bath (15 [plus or minus 1]° C.) for 1 minute. Blood samples for PK and plasma serotonin concentration will be collected again, followed by digital blood flow assessments for 30 minutes. Blood samples for PK and biomarkers will be collected again after post-cold challenge digital blood flow assessments are complete.

Biomarkers related to the mechanism of action (plasma serotonin concentration) of Compound 1 as well as vascular injury and endothelial dysfunction will be evaluated and may include plasma serotonin concentration, markers of vascular injury and endothelial dysfunction (endothelin-1, factor VIII activity, intracellular adhesion molecule 1 [ICAM-1], thromboxane B2, tissue-type plasminogen activator antigen [t-PA], plasminogen activator inhibitor-1 [PAI-1] activity, PAI-1 antigen, vascular adhesion molecule 1 (VCAM-1), vascular endothelial growth factor (VEGF), and von Willebrand factor [VWF]).

For treatment visits 2 and 3, the subject will undergo the same assessments as on Day 1. After pre-dose assessments are completed, subjects will receive study treatment as defined by their randomized treatment sequence, followed by post-dose assessments.

Following the double-blinded conduct of Part A, an unblinded evaluation of efficacy and safety results will be performed prior to proceeding to Part B. In Part B, two additional single doses of Compound 1 and placebo may be evaluated with the same study design as Part A in order to better describe the dose response. Sample size may be adjusted based on the results of Part A.

The primary outcome measure is change in digital blood flow based on (i) rewarming AUC (° C., assessed with IR thermography) up to 30 minutes following a cold challenge and (ii) reperfusion AUC (perfusion units (pu) assessed with LSCI) up to 30 minutes following a cold challenge.

Other outcome measures include:
Change in the following temperature (° C., assessed with IR thermography) and perfusion (pu, assessed with LSCI) parameters with Compound 1 versus placebo:
   Maximum reduction following a cold challenge
   Maximum recovery during the 30 minutes following a cold challenge
   AUC during the initial 2 minutes following a cold challenge
   Slope during the initial 2 minutes following a cold challenge
   Time to achieve 50% recovery from the cold challenge-induced reduction
   Time to achieve 70% recovery from the cold challenge-induced reduction
Change from predose to postdose in the following temperature (° C., assessed with IR thermography) and perfusion (pu, assessed with LSCI) parameters with Compound 1 versus placebo:
   Room temperature values
   Distal dorsal difference (DDD) (defined as the difference in measurements between the dorsum and the finger) at room temperature
Safety and tolerability of Compound 1
The concentration of plasma serotonin
The relationship between selected PD endpoints and plasma serotonin
The relationship between selected PD endpoints and the concentration of Compound 1 and/or active metabolites
VAS for pain

Inclusion criteria include:
Raynaud's phenomenon (defined as a history of digital cold sensitivity associated with color changes of cyanosis and pallor, with on average at least 5 attacks per week during the winter period) secondary to systemic sclerosis (SSc)
Agreement to use a highly effective method of birth control if the possibility of conception exists
Body mass index 18.0 to 40.0 kilograms per square meter (kg/m\hat{ }2), inclusive.

Exclusion criteria include:
Active digital ulcer(s), recent history (within 3 months of screening) of digital ulcers, or history of recurrent digital ulcerations that in the opinion of the Investigator increase the likelihood of developing a digital ulcer during the course of the study; any history of gangrene, amputations, or other critical digital ischemic event
Raynaud's phenomenon due to any cause other than SSc
Severe gastrointestinal complications related to SSc that could significantly affect Compound 1 absorption
History of gastrointestinal bleeding or active gastric or duodenal ulcers

Plasma serotonin concentration will be assessed before the start of the post-dose digital blood flow assessments and immediately after the post-dose digital blood flow assessments are completed. The relationship between plasma serotonin concentration and selected PD measures may be explored.

Blood samples for plasma PK analysis of Compound 1 and its metabolites will be collected at pre-dose, after study treatment administration (before the start of post-dose digital blood flow assessments), after the cold challenge, and again after the post-dose digital blood flow assessments are completed. Relationships between plasma concentration of selected analytes (Compound 1, its active metabolites, and/or all analytes combined) and selected PD measures may be explored.

Safety assessments will include assessment of adverse events, ECGs, vital signs, clinical chemistry and hematology, physical examinations, and concomitant medications.

Treatment with Compound 1 is expected to result in a decrease in the frequency, duration, and/or severity of Raynaud's attacks; improved digital blood flow and/or digital pressures; and/or a decrease in digital capillary dysfunction.

1. A method of treating or preventing Raynaud's, comprising:
prescribing and/or administering to an individual in need thereof 3-methoxy-N-[3-(2-methylpyrazol-3-yl)-4-(2-morpholin-4-ylethoxy)phenyl]benzamide (Compound 1), or a pharmaceutically acceptable salt, hydrate, or solvate thereof.

2. The method of claim 1, wherein the individual has primary Raynaud's.

3. The method of claim 1, wherein the individual has secondary Raynaud's.

4. The method of claim 1, wherein the individual has at least one digital ulcer.

5. The method of claim 1, wherein the individual has digital gangrene.

6. The method of claim 1, wherein the individual has systemic sclerosis.

7. The method of claim 1, wherein the individual has Raynaud's secondary to a condition selected from the group consisting of: systemic sclerosis, rheumatoid arthritis, lupus, vasculitis, atherosclerosis, cryoglobulinemia, hypothyroidism, injury, and drug reaction.

8. The method of claim 1, wherein the individual has Raynaud's secondary to systemic sclerosis.

9. The method of claim 1, wherein the individual has systemic sclerosis and at least one digital ulcer.

10. The method of claim 1, wherein Compound 1 or pharmaceutically acceptable salt, hydrate, or solvate thereof is administered orally.

11. The method of claim 1, wherein Compound 1 or pharmaceutically acceptable salt, hydrate, or solvate thereof is in the form of a tablet.

12. The method of claim 1, wherein Compound 1 or pharmaceutically acceptable salt, hydrate, or solvate thereof is in the form of a capsule.

13. The method of claim 1, wherein the individual is administered Compound 1.

14. The method of claim 1, wherein the individual is administered a pharmaceutically acceptable salt of Compound 1.

15. The method of claim 1, wherein the individual is administered an HCl salt of Compound 1.

16. The method of claim 1, wherein the treating comprises at least one of the following: a reduction in a frequency of attacks, a reduction in a duration of attacks, a reduction in a severity of attacks, a reduction in ischemic tissue injury, and a reduction in digital ulceration.

17. The method of claim 16, wherein the treating comprises a reduction in the frequency of attacks, a reduction in the duration of attacks, or a reduction in the severity of attacks.

18. The method of claim 1, wherein the preventing comprises at least one of the following: a prevention of attacks, a prevention of ischemic tissue injury, and a prevention of digital ulceration.

19. The method of claim 1, wherein about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, or about 180 mg of the Compound 1 or pharmaceutically acceptable salt, hydrate, or solvate thereof is administered per day.

20. (canceled)

21. The method of claim 1, wherein the Compound 1 or pharmaceutically acceptable salt, hydrate, or solvate thereof is administered three times daily (TID).

22. (canceled)

23. (canceled)
